(19) United States
(12) Patent Application Publication
(10) Pub. No.: US 2024/0418725 A1
(43) Pub. Date: Dec. 19, 2024

(54) METHOD FOR DETECTING AND ISOLATING CELL POPULATION CO-EXPRESSING CD45 AND EPCAM AND USE THEREOF

(71) Applicant: Qingdao Sino-Cell Biomedicine Co., Ltd., Shandong (CN)

(72) Inventors: Zhen Sun, Shandong (CN); Bin Li, Shandong (CN); Wenjing Li, Shandong (CN); Mingming Zhao, Shandong (CN); Zeyao Wang, Shandong (CN); Wenna Liu, Shandong (CN); Qing Gao, Shandong (CN)

(73) Assignee: Qingdao Sino-Cell Biomedicine Co., Ltd., Shandong (CN)

(21) Appl. No.: 18/813,645

(22) Filed: Aug. 23, 2024

Related U.S. Application Data
(63) Continuation of application No. PCT/CN2022/077693, filed on Feb. 24, 2022.

(51) Int. Cl. G01N 33/574; G01N 15/14; G01N 33/543

(52) U.S. Cl. CPC G01N 33/57492; G01N 15/14; G01N 33/54326; G01N 33/57411; G01N 33/57415; G01N 33/57423; G01N 33/57449; G01N 2015/1486; G01N 2333/70589; G01N 2333/70596

(57) ABSTRACT

The present invention relates to a method for detecting and isolating a cell population co-expressing CD45 and EpCAM and the use thereof. In the present invention, a composition or a kit comprising an anti-CD45 antibody and an anti-EpCAM antibody is used for detecting and isolating a cell population co-expressing CD45 and EpCAM from PBMCs, and is used for rapid in-vitro screening of cancer, guiding treatment of cancer, and evaluating the effectiveness of cancer treatment.

### CROSS-REFERENCE TO RELATED PATENT APPLICATIONS

This application is a continuation of International Application No. PCT/CN2022/077693, filed Feb. 24, 2022, the entire disclosure of which is incorporated herein in its entirety, including its specification, drawings, claims, and abstract.

### BACKGROUND

Cancer is a tumor that threatens human health, and its incidence and mortality are increasing rapidly worldwide. In 2018, there were 18.1 million new cases and 9.6 million cancer deaths worldwide, with lung cancer ranking first in terms of incidence and mortality (Bray et al., 2018). Lung cancer, as the leading cancer killer, has the characteristics of insidious onset, untimely diagnosis, easy metastasis and recurrence, and extremely poor prognosis. In addition, 70-75% of patients are not discovered until the advanced stage, which greatly reduces the survival rate. The development of primary tumor cells in patients is usually undetectable using conventional histopathological examination. In recent years, it has been discovered that Circulating Tumor Cells (CTCs) can be used for early diagnosis, as an auxiliary method for early screening, and can be used to identify whether patients after tumor resection need further systemic treatment.

CTC refers to the cells formed by tumor cells in the primary tumor or metastatic lesions that enter the circulating blood through active migration, invasion, or external interference that causes them to fall off (King et al., 2015; Lin et al., 2014). In 1869, Australian doctor Ashworth discovered cells similar to primary tumors in the peripheral blood of patients who died of cancer under a microscope, and first proposed the concept of CTC (Ashworth, 1869). The presence of CTCs is an important risk factor for cancer metastasis and reduced survival rate. As a target of liquid biopsy, the results of CTCs can reflect the characteristics of primary and metastatic lesions. Therefore, in the diagnosis and treatment of lung cancer, CTC detection, counting and phenotypic analysis have gradually become the hot spots and focus of current research CTCs have important value in early diagnosis, efficacy evaluation, prognosis prediction and guidance of individualized treatment and the like.

In terms of lung cancer screening, commonly used methods such as low-dose spiral CT (LDCT), tumor autoantibodies, and tumor markers cannot accurately distinguish between benign and malignant tumors alone. For example, for the most commonly used LDCT, due to the diversity of small pulmonary nodules in imaging, different diseases with the same image or the same disease with different images, makes it very difficult to distinguish between benign and malignant tumors, resulting in high false positive rate, overdiagnosis, delayed diagnosis, and follow-up reexaminations that aggravate patients' anxiety and affect their normal work and life. In addition, it also has disadvantages such as iatrogenic radiation and high screening costs. Theoretically, in patients with early-stage lung cancer, CTCs already exist in the peripheral blood even before microscopic lesions are formed in the lungs, and the difficulty lies in finding an effective detection method with high sensitivity and specificity.

After CTCs enter the peripheral blood, most of them undergo apoptosis or are phagocytosed, and only a few can escape and develop into metastases. Research has shown that a tumor with a volume of 1 cm³ (approximately 1 g wet weight) typically contains 1×10⁹ cells, and about 10⁶ cells enter the blood every day (Chang et al., 2000).

The number of CTCs in circulating blood often shows a dynamic decrease, and it is very rare when reaching peripheral blood. According to research, only one tumor cell can be detected in every 106-107 white blood cells (Paterlini-Brechot and Benali, 2007; Sleijfer et al., 2007). Therefore, it is not easy to accurately detect CTCs from tens of millions of background cells, which is the main reason that limits its clinical application. The current mainstream idea is to enrich tumor cells in peripheral blood first and then detect them. The enrichment method mainly utilizes the surface molecular markers or physical characteristics of tumor cells. Subsequent tests include real-time Quantitative Reverse-transcription PCR (qRT-PCR) and other gene-level tests; Flow Cytometry (FCM), CellSearch, CTC-chip and other cell-level tests.

Although there are many CTC isolation and enrichment technologies used in clinical research, they still generally have disadvantages such as large sampling volume, poor sensitivity, low specificity, cumbersome operation and high cost at current. For example, density gradient centrifugation requires a large amount of blood; ISET has low sensitivity; 3D microfilters are expensive and the blood needs to be diluted; the content of tumor markers in qRT-PCR method is not always positively correlated with the actual individual, since the expression levels of tumor markers vary among different CTCs; CellSearch detection process is cumbersome, etc.

### SUMMARY

The present invention provides a composition comprising an anti-CD45 antibody and an anti-EpCAM antibody and a corresponding kit, as well as the use of the composition and the kit in detecting and isolating a cell population co-expressing CD45 and EpCAM from peripheral blood mononuclear cells (PBMCs); and also provides a cell population co-expressing CD45 and EpCAM and use thereof. The present invention aims to provide a more efficient detection method, and the detected cell population can be more accurately used for cancer screening, auxiliary diagnosis, efficacy detection, etc., which solves the problems of large sampling volume and difficult separation/enrichment/identification in the current detection of CTCs.

In one aspect, the present invention provides a composition comprising an anti-CD45 antibody and an anti-EpCAM antibody.

In another aspect, the present invention further provides a kit comprising the composition of the present invention.

In another aspect, the present invention further provides use of the composition or the kit in detecting and/or isolating a cell population co-expressing CD45 and EpCAM from peripheral blood mononuclear cells.

In another aspect, the present invention further provides a method for isolating a cell population co-expressing CD45 and EpCAM, comprising: providing peripheral blood mononuclear cells; mixing the composition of the present invention or the kit of the present invention with the peripheral blood mononuclear cells, and incubating the obtained mixture; adding magnetic beads to the mixture, and incubating the mixture to obtain magnetic beads bound to a cell population co-expressing CD45 and EpCAM; and removing the magnetic beads to obtain the cell population co-expressing CD45 and EpCAM.

In another aspect, the present invention further provides a cell population co-expressing CD45 and EpCAM, which is obtained by the isolation method of the present invention.

In another aspect, the present invention further provides use of the cell population co-expressing CD45 and EpCAM in screening and/or guiding cancer treatment.

In another aspect, the present invention further provides an in vitro screening method for cancer, comprising: taking peripheral blood mononuclear cells from a subject; isolating a cell population co-expressing CD45 and EpCAM of the subject by the isolation method described in the present invention; and when the proportion of the number of cells in the cell population co-expressing CD45 and EpCAM to the number of cells in the peripheral blood mononuclear cells is higher than 0.01%, further diagnosis and/or treatment should be performed.

In another aspect, the present invention further provides another in vitro screening method for cancer, comprising: taking peripheral blood mononuclear cells from a subject; mixing the composition of the present invention or the kit of the present invention with the peripheral blood mononuclear cells, and incubating the obtained mixture; detecting a cell population co-expressing CD45 and EpCAM in the mixture; and when the proportion of the number of cells in the cell population co-expressing CD45 and EpCAM to the number of cells in the peripheral blood mononuclear cells is higher than 0.01%, further diagnosis and/or treatment should be performed.

In another aspect, the present invention further provides a method for guiding cancer treatment, comprising: taking peripheral blood mononuclear cells from a subject; isolating a cell population co-expressing CD45 and EpCAM of the subject by the isolation method described in the present invention; and further examining and/or treating the subject, when the proportion of the number of cells in the cell population co-expressing CD45 and EpCAM to the number of cells in the peripheral blood mononuclear cells is higher than 0.01%.

In another aspect, the present invention further provides another method for guiding cancer treatment, comprising: taking peripheral blood mononuclear cells from a subject; mixing the composition of the present invention or the kit of the present invention with the peripheral blood mononuclear cells, and incubating the obtained mixture; detecting a cell population co-expressing CD45 and EpCAM in the mixture; and further examining and/or treating the subject, when the proportion of the number of cells in the cell population co-expressing CD45 and EpCAM to the number of cells in the peripheral blood mononuclear cells is higher than 0.01%.

In another aspect, the present invention further provides a method for evaluating the effectiveness of cancer treatment, comprising: taking first peripheral blood mononuclear cells from a subject, wherein the subject suffers from cancer and has not received anti-cancer treatment; isolating a first cell population co-expressing CD45 and EpCAM of the subject by the isolation method described in the present invention; recording the proportion x of the number of cells in the first cell population to the number of cells in the peripheral blood mononuclear cells; taking second peripheral blood mononuclear cells from the subject, after the patient is given anti-cancer treatment; isolating a second cell population co-expressing CD45 and EpCAM of the subject by the isolation method described in the present invention; recording the proportion y of the number of cells in the second cell population to the number of cells in the peripheral blood mononuclear cells; and when y<x, the method of anti-cancer treatment for the patient is effective; when y≥x, the method of anti-cancer treatment for the patient does not meet expectations.

In another aspect, the present invention further provides another method for evaluating the effectiveness of cancer treatment, comprising: taking first peripheral blood mononuclear cells from a subject, wherein the subject suffers from cancer and has not received anti-cancer treatment; mixing the composition of the present invention or the kit of the present invention with the first peripheral blood mononuclear cells to obtain a first mixture, and incubating the first mixture; detecting a first cell population co-expressing CD45 and EpCAM in the first mixture; recording the proportion x of the number of cells in the first cell population to the number of cells in the first peripheral blood mononuclear cells; taking second peripheral blood mononuclear cells from the subject, after the patient is given anti-cancer treatment; mixing the composition of the present invention or the kit of the present invention with the second peripheral blood mononuclear cells to obtain a second mixture, and incubating the second mixture; detecting a second cell population co-expressing CD45 and EpCAM in the second mixture; recording the proportion y of the number of cells in the second cell population to the number of cells in the second peripheral blood mononuclear cells; and when y<x, the method of anti-cancer treatment for the patient is effective; when y≥x, the method of anti-cancer treatment for the patient does not meet expectations.

The present invention has the following beneficial effects: The composition or the kit provided by the present invention can detect and isolate a cell population co-expressing CD45 and EpCAM from peripheral blood mononuclear cells (PBMCs). Since the content of the cell population co-expressing CD45 and EpCAM in peripheral blood mononuclear cells in healthy people is significantly lower than that in cancer patients, the composition, the kit and the isolated cell population co-expressing CD45 and EpCAM provided by the present invention can be used for rapid in vitro screening of cancer, treatment of cancer and evaluation of the effectiveness of cancer treatment.

Accordingly, the in vitro screening method for cancer, the treatment of cancer and the evaluation method for the effectiveness of cancer treatment provided by the present invention have the advantages of low cost, easy operation and short time, and only 5-10 ml of peripheral blood is required to extract PBMCs, which is more acceptable to the subjects, thus it is conducive to the rapid screening of cancer and has practical value for early detection, early diagnosis and early treatment of cancer.

### BRIEF DESCRIPTION OF THE DRAWINGS

FIGS. 1-3 show the results of flow cytometry analysis of CD45⁺EpCAM⁺ cell populations in PBMCs in Example 1; wherein, FIG. 1 is the results of flow cytometry analysis of CD45⁺EpCAM⁺ cell populations in PBMCs of healthy volunteers, in PBMCs of lung cancer patients, and in tumor tissues of lung cancer patients; the left side of FIG. 2 is the statistical analysis results of the proportion of CD45⁺EpCAM⁺ cell populations in PBMCs of healthy volunteers and in PBMCs of lung cancer patients; the right side of FIG. 2 is the statistical analysis results of the proportion of CD45⁺EpCAM⁺ cell populations in PBMCs and tumor tissue of lung cancer patients; FIG. 3 is the results of comparative analysis between the proportions of CD45⁺EpCAM⁺ cell populations in PBMCs of healthy volunteers, in PBMCs of lung cancer patients and in tumor tissues of lung cancer patients; PBMCs of healthy volunteers: n=25; PBMCs of lung cancer patients: n=25; tumor tissue of lung cancer patients: n=25, and significant differences were analyzed by t-test.

FIGS. 4-8 show the results of flow cytometry analysis of CD45⁺EpCAM⁺ cell populations in PBMCs of healthy volunteers in the co-culture system of Example 2; wherein, FIG. 4 is the electron microscope image of exosomes secreted by HCC827 cells (voltage 100.0 kV, magnification ×60.0 k); FIG. 5 is the results of particle size analysis of exosomes secreted by HCC827 cells; FIG. 6 is the protein expression of exosomes secreted by HCC827 cells;

FIG. 7 is the results of flow cytometry analysis of the CD45⁺EpCAM⁺ cell populations in PBMCs, PBMCs after co-culture of PBMCs with HCC827 cells, PBMCs after co-culture of PBMCs with HCC827 cell culture supernatant, PBMCs after co-culture of PBMCs with 10 μg/ml exosomes, PBMCs after co-culture of PBMCs with 50 μg/ml exosomes, and PBMCs after co-culture of PBMCs with 100 μg/ml exosomes; FIG. 8 is the statistical analysis results of the proportion of CD45⁺EpCAM⁺ cell populations in PBMCs, PBMCs after co-culture of PBMCs with HCC827 cells, PBMCs after co-culture of PBMCs with HCC827 cell culture supernatant, PBMCs after co-culture of PBMCs with 10 μg/ml exosomes, PBMCs after co-culture of PBMCs with 50 μg/ml exosomes, and PBMCs after co-culture of PBMCs with 100 μg/ml exosomes; in PBMCs group, co-culture group of PBMCs with HCC827 cell, co-culture group of PBMCs with HCC827 cell culture supernatant, co-culture group of PBMCs with 10 μg/ml exosomes, co-culture group of PBMCs with 50 μg/ml exosomes, and co-culture group of PBMCs with 100 μg/ml exosomes, samples n=3 in each group, and significant differences were analyzed by t-test.

FIG. 9 shows the results of flow cytometry analysis of the CD45⁺EpCAM⁺ cell population after co-culturing PBMCs of healthy volunteers with HCC827 cells and then sorting by magnetic beads coated with anti-CD45 antibodies and magnetic beads coated with anti-EPCAM antibodies, in Example 3.

FIGS. 10-11 show the results of flow cytometry analysis of apoptosis of CD45⁺EpCAM⁺ cell population in PBMCs of healthy volunteers in the co-culture system of Example 4; wherein, FIG. 10 is the flow cytometry analysis of apoptosis of CD45⁺EpCAM⁻ and CD45⁺EpCAM⁺ cells in PBMCs, PBMCs after co-culture of PBMCs with HCC827 cells, PBMCs after co-culture of PBMCs with HCC827 cell culture supernatant, PBMCs after co-culture of PBMCs with 10 μg/ml exosomes, PBMCs after co-culture of PBMCs with 50 μg/ml exosomes, and PBMCs after co-culture of PBMCs with 100 μg/ml exosomes; FIG. 11 is the statistical analysis of apoptosis of CD45⁺EpCAM⁻ and CD45⁺EpCAM⁺ cells in PBMCs, PBMCs after co-culture of PBMCs with HCC827 cells, PBMCs after co-culture of PBMCs with HCC827 cell culture supernatant, PBMCs after co-culture of PBMCs with 10 μg/ml exosomes, PBMCs after co-culture of PBMCs with 50 μg/ml exosomes, and PBMCs after co-culture of PBMCs with 100 μg/ml exosomes; in PBMCs group, co-culture group of PBMCs with HCC827 cell, co-culture group of PBMCs with HCC827 cell culture supernatant, co-culture group of PBMCs and 10 μg/ml exosomes, co-culture group of PBMCs with 50 μg/ml exosomes, and co-culture group of PBMCs with 100 μg/ml exosomes, samples n=3 in each group, and significant differences were analyzed by t-test.

FIG. 12 shows the results of preoperative serological tests of lung cancer patients in Example 5; wherein, lung cancer patients: n=25.

FIG. 13 shows the proportion of CD45⁺EpCAM⁺ cell population in PBMCs of lung cancer patients and the analysis results of the content of carcino-embryonic antigen (CEA) in the serum of patients in Example 5; wherein, lung cancer patients: n=25.

### DETAILED DESCRIPTION

The present invention will be further described below in conjunction with the examples, but these examples do not constitute any limitation to the present invention.

The present invention aims to solve the problems of large sampling volume and difficult separation/enrichment/identification in the existing detection of CTCs.

In this field, CTCs are usually used to determine the prognosis of cancer treatment or the spread of tumors. In the detection process of CTCs, some cell molecular markers of non-cancer origin are often used as parameters for exclusion. CD45 molecule is specifically expressed on all white blood cells. In order to avoid the background and other adverse factors brought by the existence of white blood cells for further analysis, common CTC tests, such as CellSearch, exclude CD45+ cells from the cell population for CTC analysis and detection. There are also specific studies on how to efficiently remove white blood cells to improve the recovery rate of CTCs. However, the inventors of the present invention found that CD45+EpCAM+ cells existed in 100% of lung cancer tumor tissues. As a common technical understanding in the field, white blood cells all express CD45 but do not express EpCAM; and cells that express EpCAM usually do not express CD45, and therefore the existence of cells that co-express CD45 and EpCAM is usually not recognized. After further confirmation and analysis by the inventors, it has been determined that such cells co-expressing CD45 and EpCAM exist, and it is speculated that the formation of these CD45+EpCAM+ cells may originate from the fusion of exosomes secreted by tumor cells and white blood cells. At the same time, it also has been found that CD45+EpCAM+ cells also exist in the peripheral blood of tumor patients, while the content of CD45+EpCAM+ cells in PBMCs of healthy people is significantly lower than that of tumor patients (such as lung cancer patients). Therefore, CD45+EpCAM+ cells in PBMCs can be detected or isolated for in vitro screening, treatment or evaluation of treatment effectiveness of cancer.

Based on this, in order to obtain the CD45+EpCAM+ cells, the present invention provides a composition comprising an anti-CD45 antibody and an anti-EpCAM antibody.

Specifically, the anti-CD45 antibody and the anti-EpCAM antibody applicable to the present invention may be various anti-CD45 antibodies and anti-EpCAM antibodies known in the art. For example, commercially available anti-CD45 antibodies and anti-EpCAM antibodies may be used to implement the present invention, or the anti-CD45 antibody and the anti-EpCAM antibody may be prepared using techniques known in the art (such as hybridoma technology).

In some embodiments, the anti-CD45 antibody is a rat anti-human CD45 monoclonal antibody, a mouse anti-human CD45 monoclonal antibody, a sheep anti-human CD45 monoclonal antibody or a rabbit anti-human CD45 monoclonal antibody. Preferably, the affinity of the anti-CD45 monoclonal antibody is greater than 1.0×10−11 mol/L.

In some embodiments, the anti-EpCAM antibody is selected from a rat anti-human EpCAM monoclonal antibody, a mouse anti-human EpCAM monoclonal antibody, a sheep anti-human EpCAM monoclonal antibody, and a rabbit anti-human EpCAM monoclonal antibody. Preferably, the affinity of the anti-EpCAM antibody is greater than or equal to 2.69×10−10 mol/L.

In some embodiments, the anti-EpCAM antibody is a polyclonal antibody and/or the anti-CD45 antibody is a polyclonal antibody.

In some embodiments, the mass ratio of the anti-EpCAM antibody to the anti-CD45 antibody is 1:8.3.

In some embodiments, the anti-EpCAM antibody and the anti-CD45 antibody are conjugated with biotin or fluorescein, respectively, and the fluorescein includes but is not limited to fluorescein isothiocyanate (FITC), phycoerythrin (PE), allophycocyanin (APC), and peridinin chlorophyll protein (PerCP), etc. Wherein, when the antibody is conjugated with biotin, the antibody can be bound to magnetic beads conjugated with streptavidin to achieve isolation, detection, and subsequent further uses. When the antibody is conjugated with fluorescein, it can be used to directly achieve the detection of CD45+EpCAM+ cell population by flow cytometry and for subsequent further uses.

It is understood that the composition provided by the present invention may also comprise a suitable solvent, including but not limited to PBS, EDTA, BSA, etc.

Accordingly, the present invention further provides a kit comprising the composition of the present invention.

In some embodiments, the kit also comprises magnetic beads. The magnetic beads used in the present invention may be various magnetic beads for cell separation or enrichment known in the art. Preferably, the magnetic beads are conjugated with streptavidin, thereby binding to the biotin conjugated on the anti-CD45 antibody and the anti-EpCAM antibody in the composition, so that the CD45+EpCAM+ cells bound to the anti-CD45 antibody and the anti-EpCAM antibody are isolated.

The composition or the kit provided by the present invention can be used to isolate a cell population co-expressing CD45 and EpCAM (CD45+EpCAM+) from peripheral blood mononuclear cells.

In some embodiments, the peripheral blood mononuclear cells are derived from tumor patients. The tumor includes but is not limited to lung cancer, breast cancer, ovarian cancer, cervical cancer, etc.; preferably, the tumor is lung cancer.

Accordingly, the present invention provides the use of anti-CD45 antibodies and anti-EpCAM antibodies, the composition as described in the present invention or the kit as described in the present invention in detecting and/or isolating a cell population co-expressing CD45 and EpCAM.

In some embodiments, when the anti-EpCAM antibody is a polyclonal antibody and/or the anti-CD45 antibody is a polyclonal antibody, polyclonal antibodies of different species may also be used to implement the detection and/or isolation of a cell population co-expressing CD45 and EpCAM. Specifically, in an example of the anti-CD45 polyclonal antibody, a sheep anti-human CD45 polyclonal antibody may be used as a primary antibody to bind to CD45; a rabbit anti-sheep antibody conjugated with biotin or fluorescein may be used as a secondary antibody to bind to the primary antibody, thereby realizing the detection and isolation of a cell population expressing CD45. As the primary antibody, the above sheep anti-human CD45 polyclonal antibody may also be replaced by anti-human CD45 polyclonal antibodies from other species, including but not limited to rat anti-human CD45 polyclonal antibody, mouse anti-human CD45 polyclonal antibody, and rabbit anti-human CD45 polyclonal antibody. The secondary antibody includes but is not limited to rabbit anti-sheep antibody, mouse anti-sheep antibody, rat anti-sheep antibody, goat anti-rabbit antibody, etc.

Accordingly, the present invention further provides a method for isolating a cell population co-expressing CD45 and EpCAM, comprising: (11) providing peripheral blood mononuclear cells; (12) mixing the composition of the present invention or the kit of the present invention with the peripheral blood mononuclear cells, and incubating the obtained mixture; (13) adding magnetic beads to the mixture, and incubating the mixture to obtain magnetic beads bound to a cell population co-expressing CD45 and EpCAM; and (14) removing the magnetic beads to obtain the cell population co-expressing CD45 and EpCAM.

Specifically, in step (11), the peripheral blood mononuclear cells (PBMCs) may be obtained by methods known in the art.

In step (12), the method of mixing the composition or the kit with the PBMCs peripheral blood mononuclear cells and the method of incubating the mixture are methods known in the art, and the time, temperature and other conditions required for incubation may be determined according to actual situation. In some embodiments, the incubation temperature is 4° C. and the incubation time is 20 min.

In step (13), the magnetic beads are added to the mixture, and the magnetic beads are conjugated with streptavidin and can bind to the biotin conjugated on the anti-CD45 antibody and the anti-EpCAM antibody in the composition, thereby obtaining magnetic beads of a cell population co-expressing CD45 and EpCAM.

It is understood that when the kit is used to isolate the cell population co-expressing CD45 and EpCAM and the kit already comprises the magnetic beads, the magnetic beads and the composition are placed separately in the kit. In step (12), the composition in the kit is first mixed and incubated with the peripheral blood mononuclear cells to obtain a mixture; and in step (13), the magnetic beads in the kit are then incubated with the mixture.

In the present invention, when isolating the cell population co-expressing CD45 and EpCAM, the CD45+EpCAM+ cell population may be isolated at one time, or a stepwise method may be adopted to first isolate a EpCAM+ cell population and then separate a CD45+ cell population from the EpCAM+ cell population to finally isolate and obtain the CD45+EpCAM+ cell population, or vice versa. That is, in the present invention, there is no special requirements for the order of isolating the CD45+ cell population and the EpCAM+ cell population, which can be performed simultaneously or sequentially. However, since the number of CD45+ cells in peripheral blood mononuclear cells is usually greater than that of EpCAM+ cells, it is preferred to isolate the CD45+ cell population first in the stepwise isolation method. In an embodiment, when the cell population co-expressing CD45 and EpCAM is isolated from the mixture using the magnetic beads, the CD45+ cells in the mixture are first isolated using magnetic beads, and then the EpCAM+ cells are isolated from the CD45+ cells using magnetic beads.

In step (14), the method for removing the magnetic beads may employ methods known in the art, including but not limited to performing magnetic separation on a magnetic grate to remove the magnetic beads.

Accordingly, the present invention further provides a cell population co-expressing CD45 and EpCAM, which is obtained by the isolation method of the present invention. The method for isolating a cell population co-expressing CD45 and EpCAM provided by the present invention can isolate the cell population co-expressing CD45 and EpCAM quickly and efficiently, and the cell population co-expressing CD45 and EpCAM can be used for screening and/or guiding cancer treatment.

The use of the cell population co-expressing CD45 and EpCAM provided by the present invention includes, but is not limited to, in vitro screening of cancer, guiding cancer treatment, and evaluation of the effectiveness of cancer treatment methods. The above-mentioned use of the cell population co-expressing CD45 and EpCAM of the present invention has several advantages: first, the sampling volume is reduced. The present invention only needs to draw 5-10 ml of peripheral blood from a subject to extract PBMCs, which is more acceptable to the subject and solves the problem of large sampling volume in CTC detection. Second, it is easy to operate and takes a short time. When the present invention isolates the cell population co-expressing CD45 and EpCAM and uses it for the above uses, it can be accomplished by flow cytometry without complicated steps or instruments; and compared with CellSearch and other detection methods, the present invention significantly reduces the detection cost, simplifies technical operations. Third, it has higher sensitivity. The inventors of the present invention have found through research that the number of CD45+EpCAM+ cells is greater than that of CD45-EpCAM+ cells, when using existing CTC enrichment or detection methods, this part of the suggestive CD45+EpCAM+ cells will be removed, and then resulting in greatly reduced sensitivity in detection, screening or diagnosis. By using the content of the cell population co-expressing CD45 and EpCAM as an indicator, the present invention has sensitivity that is not only higher than CTCs, but also higher than CEA (about twice), the most sensitive indicator in current lung cancer screening.

In some embodiments, the cancer that the cell population co-expressing CD45 and EpCAM is used for screening, treatment, and evaluation, includes but is not limited to lung cancer, breast cancer, ovarian cancer, cervical cancer, etc.; lung cancer is preferred.

Accordingly, the present invention further provides an in vitro screening method for cancer, comprising: (21) taking peripheral blood mononuclear cells from a subject; (22) isolating a cell population co-expressing CD45 and EpCAM of the subject by the isolation method described in the present invention; and (23) when the proportion of the number of cells in the cell population co-expressing CD45 and EpCAM to the number of cells in the peripheral blood mononuclear cells is higher than 0.01%, further diagnosis and/or treatment should be performed.

Specifically, in step (21), the peripheral blood mononuclear cells of the subject may be obtained by methods known in the art. In some embodiments, the subject is an individual suspected of suffering cancer, particularly an individual suspected of suffering lung cancer or an individual at high risk of lung cancer.

In step (22), the method for isolating a cell population co-expressing EpCAM and CD45 of the subject by the isolation method of the present invention is the same as described in the above steps (11)-(14).

In step (23), according to the experimental tests of the present invention, the proportion of the number of CD45+EpCAM+ cells in the PBMCs of healthy people accounts for 0.01% on average. When the proportion of the number of cells in the CD45+EpCAM+ cell population to the number of PBMCs is higher than 0.01%, the subject is suspected of suffering cancer, and the probability is higher than 88%.

The method provided by the present invention such as steps (21)-(23) is to first isolate a CD45+EpCAM+ cell population by magnetic beads, and then use the CD45+EpCAM+ cell population for screening. In addition, the present invention may also be used for screening more quickly and easily by flow cytometry. Accordingly, the present invention further provides another in vitro screening method for cancer, comprising: (31) taking peripheral blood mononuclear cells from a subject; (32) mixing the composition of the present invention or the kit of the present invention with the peripheral blood mononuclear cells, and incubating the obtained mixture; (33) detecting a cell population co-expressing CD45 and EpCAM in the mixture; and (34) when the proportion of the number of cells in the cell population co-expressing CD45 and EpCAM to the number of cells in the peripheral blood mononuclear cells is higher than 0.01%, further diagnosis and/or treatment should be performed.

Specifically, in step (31), the peripheral blood mononuclear cells of the subject may be obtained by methods known in the art. In some embodiments, the subject is an individual suspected of suffering cancer, particularly an individual suspected of suffering lung cancer or an individual at high risk of lung cancer.

In step (32), the composition of the present invention or the kit of the present invention is mixed with the peripheral blood mononuclear cells and incubated, so that the anti-CD45 antibody and the anti-EpCAM antibody co-label the CD45+EpCAM+ cell population in the PBMCs.

In step (33), the cell population co-expressing CD45 and EpCAM may be detected by using conventional detection methods in the art, such as flow cytometry, immunofluorescence staining, western blotting, real-time quantitative reverse transcription PCR, etc. In some embodiments, it is preferred to detect by flow cytometry. Specifically, the cells in the mixture are stained to detect the CD45+EpCAM+ cell population. The cell staining and specific detection steps of flow cytometry may be carried out by methods known in the art.

Step (34) is the same as step (23) described above and will not be repeated here.

The method of steps (31)-(34) provided by the present invention preferably uses flow cytometry to directly obtain data on the proportion of CD45+EpCAM+ cell population to PBMCs, which can obtain results more directly and quickly, and requires less sample volume of PBMCs and has lower requirements for experimental equipments, and is of great value in clinical rapid screening and auxiliary diagnosis.

Accordingly, the present invention further provides a method for guiding cancer treatment, comprising: (41) taking peripheral blood mononuclear cells from a subject; (42) isolating a cell population co-expressing CD45 and EpCAM of the subject by the isolation method described in the present invention; and (43) further examining and/or treating the subject, when the proportion of the number of cells in the cell population co-expressing CD45 and EpCAM to the number of cells in the peripheral blood mononuclear cells is higher than 0.01%.

Specifically, in step (41), the peripheral blood mononuclear cells of the subject may be obtained by methods known in the art. In some embodiments, the subject is an individual suspected of suffering cancer, particularly an individual suspected of suffering lung cancer or an individual at high risk of lung cancer.

Step (42) is the same as step (22) described above and will not be repeated here.

In step (43), when the proportion of the number of cells in the cell population co-expressing CD45 and EpCAM to the number of cells in the peripheral blood mononuclear cells is higher than 0.01%, the subject is suspected of suffering cancer, and the probability is higher than 88%, and further diagnosis is required to confirm that the subject suffers cancer. After the diagnosis is confirmed, the subject may be treated with corresponding cancer treatment. The treatment described in the present invention is a method known in the art, including but not limited to chemotherapy, immunotherapy and/or radiotherapy.

Similarly, the cancer treatment method of the present invention may also be a treatment after diagnosis and confirmation based on the data of the proportion of CD45+EpCAM+ cell population to PBMCs directly obtained by flow cytometry. Accordingly, the present invention further provides another method for guiding cancer treatment, comprising: (51) taking peripheral blood mononuclear cells from a subject; (52) mixing the composition of the present invention or the kit of the present invention with the peripheral blood mononuclear cells, and incubating the obtained mixture; (53) detecting a cell population co-expressing CD45 and EpCAM in the mixture; and (54) when the proportion of the number of cells in the cell population co-expressing CD45 and EpCAM to the number of cells in the peripheral blood mononuclear cells is higher than 0.01%, further diagnosis is required for the subject.

Specifically, step (51) is the same as step (31) described above, step (52) is the same as step (32) described above, and step (53) is the same as step (33) described above, and will not be repeated here.

In step (54), when the proportion of the number of cells in the cell population co-expressing CD45 and EpCAM to the number of cells in the peripheral blood mononuclear cells is higher than 0.01%, the subject is suspected of suffering cancer, and the probability is higher than 88%, and further diagnosis is required to confirm that the subject suffers cancer. After the diagnosis is confirmed, the subject may be treated with corresponding cancer treatment.

The present invention further provides a method for evaluating the effectiveness of cancer treatment, comprising: (61) taking first peripheral blood mononuclear cells from a subject, wherein the subject suffers from cancer and has not received anti-cancer treatment; (62) isolating a first cell population co-expressing CD45 and EpCAM of the subject by the isolation method described in the present invention; (63) recording the proportion x of the number of cells in the first cell population to the number of cells in the peripheral blood mononuclear cells; (64) taking second peripheral blood mononuclear cells from the subject, after the patient is given anti-cancer treatment; (65) isolating a second cell population co-expressing CD45 and EpCAM of the subject by the isolation method described in the present invention; (66) recording the proportion y of the number of cells in the second cell population to the number of cells in the peripheral blood mononuclear cells; and (67) when y<x, the method of anti-cancer treatment for the patient is effective; when y≥x, the method of anti-cancer treatment for the patient does not meet expectations.

The evaluation method provided by the present invention is to detect the proportion of CD45+EpCAM+ cell population in PBMCs of a cancer patient before and after anti-cancer treatment respectively, and then compare the data before and after treatment to determine whether the proportion of CD45+EpCAM+ cell population in PBMCs is reduced. If it is reduced, it indicates that the anti-cancer treatment is effective; if it is not reduced, it indicates that the anti-cancer treatment has not met expectations. The evaluation method provided by the present invention can be used to evaluate whether different treatment methods, different drugs or combination thereof, different administration methods, different doses, different dosage forms and the like are effective for cancer treatment of a specific individual when anti-cancer treatment is given to the specific individual.

Specifically, in step (61), the peripheral blood mononuclear cells of the subject may be obtained by methods known in the art, and the subject is an individual who has been diagnosed with cancer, preferably an individual diagnosed with lung cancer.

In some embodiments, the number of cells in the first peripheral blood mononuclear cells obtained in step (61) is set equal to the number of cells in the second peripheral blood mononuclear cells obtained in step (64) to improve the accuracy of the evaluation result.

Similarly, the present invention may also perform the evaluation based on the data of the proportion of CD45+EpCAM+ cell population to PBMCs directly obtained by flow cytometry. Accordingly, the present invention further provides another method for evaluating the effectiveness of cancer treatment, comprising: (71) taking first peripheral blood mononuclear cells from a subject, wherein the subject suffers from cancer and has not received anti-cancer treatment; (72) mixing the composition of the present invention or the kit of the present invention with the first peripheral blood mononuclear cells to obtain a first mixture, and incubating the first mixture; (73) detecting a first cell population co-expressing CD45 and EpCAM in the first mixture; (74) recording the proportion x of the number of cells in the first cell population to the number of cells in the first peripheral blood mononuclear cells; (75) taking second peripheral blood mononuclear cells from the subject, after the patient is given anti-cancer treatment; (76) mixing the composition of the present invention or the kit of the present invention with the second peripheral blood mononuclear cells to obtain a second mixture, and incubating the second mixture; (77) detecting a second cell population co-expressing CD45 and EpCAM in the second mixture; (78) recording the proportion y of the number of cells in the second cell population to the number of cells in the second peripheral blood mononuclear cells; and (79) when y<x, the method of anti-cancer treatment for the patient is effective; when y≥x, the method of anti-cancer treatment for the patient does not meet expectations.

Specifically, in step (71), the peripheral blood mononuclear cells of the subject may be obtained by methods known in the art, and the subject is an individual who has been diagnosed with cancer, preferably an individual diagnosed with lung cancer.

In some embodiments, the number of cells in the first peripheral blood mononuclear cells obtained in step (71) is set equal to the number of cells in the second peripheral blood mononuclear cells obtained in step (75) to improve the accuracy of the evaluation result.

In steps (73) and (77), the cell population co-expressing CD45 and EpCAM may be detected by using conventional methods for detecting cell markers in the art. In some embodiments, it is preferred to detect by flow cytometry. Specifically, the cells in the mixture are stained to detect the CD45+EpCAM+ cell population. Cell staining and the specific detection steps of flow cytometry may be carried out by methods known in the art.

In order to enable the above implementation details and operations of the invention clearly understood by those skilled in the art, and to significantly reflect the improved performance of the method for detecting and isolating a cell population co-expressing CD45 and EpCAM and use thereof in examples of the invention, the above technical solution is illustrated by multiple examples below.

The experimental methods used in the following examples are conventional methods unless otherwise specified.

Materials, reagents, etc. used in the following examples can all be obtained from commercial sources unless otherwise specified.

### Example 1

1. Flow cytometry detection of the expression of CD45 and EpCAM on cells of lung cancer tumor tissue: (101) tumor tissue from lung cancer patients was obtained by minimally invasive surgery, placed in a culture dish with a diameter of 10 cm, and washed once with normal saline; (102) the tumor tissue was cut into 1 mm3 tissue blocks by ophthalmic scissors or a scalpel; (103) an appropriate amount of normal saline was added, and then the minced tumor tissue was transferred to a 50 ml centrifuge tube and centrifuged at 400 g for 5 min; (104) after centrifugation, the supernatant was discarded, 1 ml of normal saline was added to resuspend the cells, then normal saline was added to 50 ml, and mixed by inverting; (105) the cells were filtered by a 70 μm filter into a new 50 ml centrifuge tube to obtain a cell suspension; (106) appropriate amount of cells were taken for cell counting and then the cell suspension was centrifuged at 400 g for 5 min; (107) after centrifugation, the supernatant was discarded, 1 ml of normal saline was added to resuspend the cells, and 0.5×10⁶ cells were taken for flow cytometry staining; (108) centrifugation was performed at 400 g for 10 min; (109) after centrifugation, the supernatant was discarded and the cell pellet was resuspended in 100 μl of PBS containing 2% fetal bovine serum. The Fc part of the antibody will bind to the Fc receptors on the surface of white blood cells or tumor cells. Therefore, in order to avoid the generation of background signals or the appearance of nonspecific staining caused by such nonspecific binding, Fc receptor blockers were added to each sample and incubated at room temperature for 10 min; (110) the cells were divided into two groups, the all-negative group and the group to be tested, with a cell volume of 50 μl in each group, i.e., 2.5×105 viable cells; (111) 1.25 μl of APC-EpCAM antibody and Alexa Fluor 700-CD45 antibody were added to the cells in the group to be tested and incubated in a refrigerator at 4° C. for 20 min; (112) after the incubation, 1.25 μl of PerCP-7AAD dye was added to the cells in the test group and incubated at room temperature for 10 min; (113) after the incubation, 1 ml of PBS containing 2% fetal bovine serum was added to each tube of cells and centrifuged at 400 g for 5 min; (114) after centrifugation, the supernatant was discarded and 200 μl of PBS containing 2% fetal bovine serum was added to resuspend the cells and mixed thoroughly; (115) cells were filtered through a 70 μm filter and then analyzed by flow cytometry.

2. Flow cytometry detection of the expression of CD45 and EpCAM on peripheral blood mononuclear cells (PBMCs): (201) 5-10 ml of peripheral blood was drawn from healthy volunteers and lung cancer patients into anticoagulant tubes; (202) the anticoagulant tube containing the blood sample was disinfected with 75% alcohol and placed in a super clean bench, the lid of the anticoagulant tube was opened and the peripheral blood was transferred to a 50 ml centrifuge tube and centrifuged at 650 g for 10 min; (203) after centrifugation, the blood was separated into two layers, and the upper plasma was discarded using a Pasteur pipette; (204) an equal volume of normal saline was added to the precipitate and mixed thoroughly by pipetting with a Pasteur pipette; (205) mononuclear cells in peripheral blood include lymphocytes and monocytes and the like, and their volume, morphology and density are different from other cells. Cells such as red blood cells and white blood cells have the highest density, followed by lymphocytes and monocytes, and platelets have the lowest density. The sample density separation solution was used to separate different cells in peripheral blood through density separation. The sample density separation solution was prepared, the volume of which was consistent with the volume of the mixture of the precipitate and normal saline; (206) the mixture of precipitate and normal saline were slowly added into the sample density separation solution with a Pasteur pipette, and the two could not be mixed; (207) centrifugation was performed at 650 g for 10 min; (208) after centrifugation, the mixture was divided into 4 layers, and the second layer was taken out with a Pasteur pipette into a new 50 ml centrifuge tube, and normal saline was added to 50 ml; (209) centrifugation was performed at 600 g for 10 min and the supernatant was discarded; (210) an appropriate amount of normal saline was added and mixed thoroughly by pipetting, cells were counted and 0.5×106 cells were taken from each sample for flow cytometry staining; (211) centrifugation was performed at 400 g for 10 min; (212) after centrifugation, the supernatant was discarded and the cell pellet was resuspended in 100 μl of PBS containing 2% fetal bovine serum. The Fc part of the antibody will bind to the Fc receptors on the surface of white blood cells or tumor cells. Therefore, in order to avoid the generation of background signals or the appearance of nonspecific staining caused by such nonspecific binding, Fc receptor blockers were added to each sample and incubated at room temperature for 10 min; (213) the cells of each sample were divided into two groups, the all-negative group and the group to be tested, with a cell volume of 50 μl in each group, i.e., 2.5×105 viable cells; (214) 1.25 μl of APC-EpCAM antibody and Alexa Fluor 700-CD45 antibody were added to the cells in the group to be tested and incubated in a refrigerator at 4° C. for 20 min; (215) after the incubation, 1.25 μl of PerCP-7AAD dye was added to the cells in the test group and incubated at room temperature for 10 min; (216) after the incubation, 1 ml of PBS containing 2% fetal bovine serum was added to each tube of cells and centrifuged at 400 g for 5 min; (217) after centrifugation, the supernatant was discarded and 200 μl of PBS containing 2% fetal bovine serum was added to resuspend the cells and mixed thoroughly; (218) cells were filtered through a 70 μm filter and then analyzed by flow cytometry.

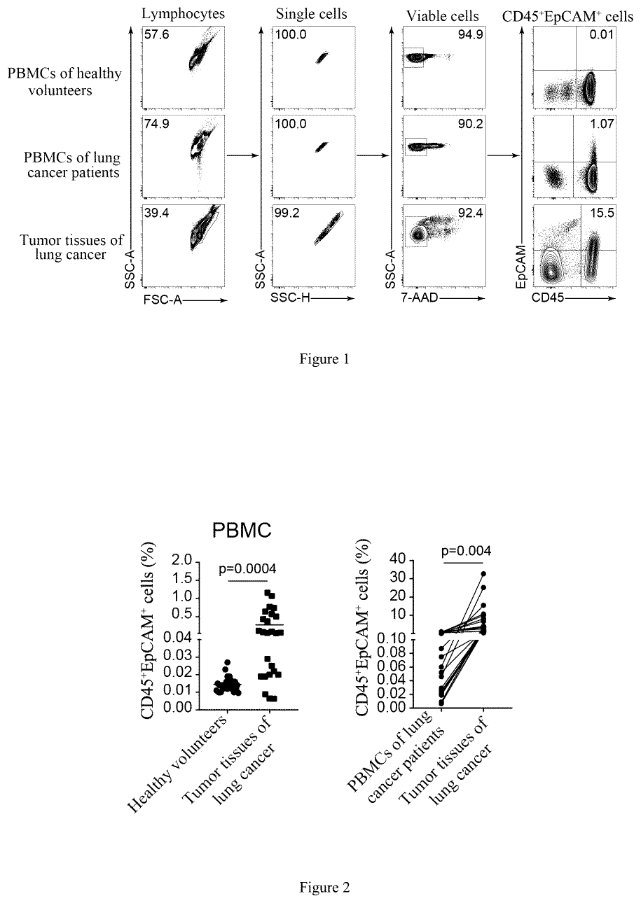

After the flow cytometry staining of PBMCs of healthy volunteers, PBMCs of lung cancer patients and single cell suspension of lung cancer tumor tissue was completed, Beckman flow cytometer was used for analysis, and the results are shown in FIGS. 1-3. FIG. 1 is a flow cytometric graph, shows that compared with PBMCs of healthy volunteers, the proportion of CD45+EpCAM+ cells in PBMCs of lung cancer patients and in the tumor tissue was increased significantly. FIG. 2 is a statistical graph, shows that compared with PBMCs of healthy volunteers, the proportion of CD45+EpCAM+ cells in PBMCs of lung cancer patients and in the tumor tissue was increased significantly, which is consistent with the flow cytometry results. FIG. 3 is a statistical graph, and it can be seen from the results that the proportion of CD45+EpCAM+ cells in the tumor tissue of lung cancer patients was significantly increased compared with the corresponding PBMCs; there is a certain linear relationship between the proportion of CD45+EpCAM+ cells in the tumor tissue of lung cancer patients and that in PBMCs, patients with a high proportion of CD45+EpCAM+ cells in lung cancer tumor tissues have a higher proportion of CD45+EpCAM+ cells in PBMCs than that of healthy volunteers.

### Example 2

Study on the causes of the formation of CD45+EpCAM+ cells in peripheral blood mononuclear cells (PBMCs).

1. Extraction of exosomes from HCC827 cells: (301) when the confluency of HCC827 cells reached 80-90%, the cells were passaged and cultured with RPMI 1640 complete medium containing exosome-free serum for 48-72 h, and the cell culture supernatant was collected; (302) cells were removed by centrifugation at 300-500 g, 4° C. for 5 min; (303) after centrifugation, the cell culture supernatant was transferred to a new centrifuge tube and centrifuged at 2,000 g, 4° C. for 10 min to remove cell debris; (304) after centrifugation, the cell culture supernatant was transferred to a new centrifuge tube and centrifuged at 10,000 g, 4° C. for 30 min to remove apoptotic bodies; (305) after centrifugation, the cell culture supernatant was transferred to a new centrifuge tube and centrifuged at 120,000 g, 4° C. for 70 min, and the supernatant was discarded and the precipitate was collected; (306) after 1 ml of PBS was added to resuspend the precipitate, PBS was added to 50 ml, and then centrifuged again at 120,000 g, 4° C. for 70 min, and the precipitate was the extracted exosomes; (307) after 1 ml of PBS was added to resuspend the precipitate, the resuspension solution was transferred to a 1.5 ml centrifuge tube and stored at −80° C.

2. Transmission electron microscopy observation of exosome samples: (308) 10 μl of exosomes were taken; (309) 10 μl of sample was pipetted and dropped on the copper grid to precipitate for 1 min, and the floating liquid was removed with filter paper; (310) 10 μl of uranyl acetate was dropped on the copper grid to precipitate for 1 min, and the floating liquid was removed with filter paper; (311) the precipitate was dried at room temperature for several minutes; (312) the electron microscope was used to detect and image the samples at 100 kV, and the results of transmission electron microscope imaging were obtained.

3. Particle size analysis of exosome sample: (313) 10 μl of exosomes was taken and diluted to 30 μl; (314) the instrument performance was tested with standard sample, only after the test was qualified, the exosome sample could be loaded; it should be noted that gradient dilution was required to avoid clogging of the injection needle by the sample; (315) once the sample was tested, the particle size information of the exosomes detected by the instrument could be obtained.

4. Detection of markers of exosome samples: day 1: (316) the BCA method was used to measure the concentration of exosome protein, and the protein concentration of the sample to be tested was calculated based on the standard curve; (317) exosomes containing 25 μg of protein were taken and the volume was made up to 100 μl with PBS containing 0.1% BSA; (318) 20 μl of the oscillated and mixed magnetic beads were taken and transferred to a suitable centrifuge tube; (319) 200 μl of PBS containing 0.1% BSA was added to the centrifuge tube containing the magnetic beads and mixed thoroughly; (320) the centrifuge tube was placed on a magnetic grate for 1 min and the supernatant was discarded; (321) the prepared exosomes were transferred to the exosomes containing magnetic beads and mixed thoroughly; (322) the mixture of the exosomes and magnetic beads was placed on a sample mixer and incubated in a refrigerator at 4° C. overnight.

Day 2: (323) after the incubation, centrifugation was performed at 13,000 g, 4° C. for 3-5 s, the supernatant was discarded, 300 μl of PBS containing 0.1% BSA was added and mixed gently with a pipette; (324) the centrifuge tube was placed on a magnetic grate for 1 min and the supernatant was discarded; 400 μl of PBS containing 0.1% BSA was added and mixed gently with a pipette; (325) the centrifuge tube was placed on a magnetic grate for 1 min and the supernatant was discarded; (326) 300 μl of PBS containing 0.1% BSA was added to resuspend the exosomes bound to magnetic beads, at this time, the concentration of the exosomes bound to magnetic beads was equivalent to 1×106 cells; (327) 1.5 μl of PE-CD63 antibody was transferred to a suitable centrifuge tube; (328) 100 μl of exosomes bound to magnetic beads was transferred to the centrifuge tube containing the antibody and mixed gently with a pipette; (329) the centrifuge tube was placed on a shaker at 1,000 rpm and incubated at room temperature for 45-60 min; (330) after the incubation, 300 μl of PBS containing 0.1% BSA was added and mixed gently with a pipette; (331) the centrifuge tube was placed on a magnetic grate for 1 min and the supernatant was discarded; (332) 100 μl of PBS containing 0.1% BSA was added to resuspend the exosomes bound to magnetic beads, mixed gently with a pipette, and analyzed by flow cytometry.

5. Co-culture experiment of PBMCs of healthy volunteers with HCC827 cells: (333) single cell suspensions of PBMCs and HCC827 cells were prepared and the cells were counted; (334) experimental groups: \textcircled{1} PBMC group: PBMC cells (1×106) were resuspended in 2 ml of RPMI 1640 complete medium containing exosome-free serum and then added to a 6-well plate; \textcircled{2} co-culture group of PBMCs with HCC827 cells: PBMCs and HCC827 cells (PBMC: HCC827=1:3) were resuspended in 2 ml of RPMI 1640 complete medium containing exosome-free serum and then added to a 6-well plate; \textcircled{3} co-culture group of PBMCs with HCC827 cell culture supernatant: PBMC cells (1×106) were resuspended in 2 ml of HCC827 cell culture supernatant and then added to a 6-well plate; \textcircled{4} co-culture group of PBMCs with HCC827 cell exosomes: PBMC cells (1×106) were resuspended in 2 ml of RPMI 1640 complete medium containing exosome-free serum, and then 10 μg/ml exosomes were added, resuspended and mixed, and then added to a 6-well plate; \textcircled{5} co-culture group of PBMCs with HCC827 cell exosomes: PBMC cells (1×106) were resuspended in 2 ml of RPMI 1640 complete medium containing exosome-free serum, and then 50 μg/ml exosomes were added, resuspended and mixed, and then added to a 6-well plate; \textcircled{6} co-culture group of PBMCs with HCC827 cell exosomes: PBMC cells (1×106) were resuspended in 2 ml of RPMI 1640 complete medium containing exosome-free serum cells, then added 100 μg/ml exosomes, resuspended and mixed, and added to a 6-well plate; (335) after the cells were placed in an incubator at 37° C. and incubated for 24 h, the cells were observed and then collected; (336) centrifugation was performed at 400 g for 10 min; (337) after centrifugation, the supernatant was discarded and the cell pellet was resuspended in 100 μl of PBS containing 2% fetal bovine serum. The Fc part of the antibody will bind to the Fc receptors on the surface of white blood cells or tumor cells. Therefore, in order to avoid the generation of background signals or the appearance of nonspecific staining caused by such nonspecific binding, Fc receptor blockers were added to each sample and incubated at room temperature for 10 min; (338) the cells of each sample were divided into two groups, the all-negative group and the group to be tested, with a cell volume of 50 μl in each group, i.e., 5×105 viable cells; (339) 2.5 μl of APC-EpCAM antibody and Alexa Fluor 700-CD45 antibody were added to the cells in the group to be tested and incubated in a refrigerator at 4° C. for 20 min; (340) after the incubation, 2.5 μl of PerCP-7AAD dye was added to the cells in the test group and incubated at room temperature for 10 min; (341) after the incubation, 1 ml of PBS containing 2% fetal bovine serum was added to each tube of cells and centrifuged at 400 g for 5 min; (342) after centrifugation, the supernatant was discarded and 200 μl of PBS containing 2% fetal bovine serum was added to resuspend the cells and mixed thoroughly; (343) cells were filtered through a 70 μm filter and then analyzed by flow cytometry.

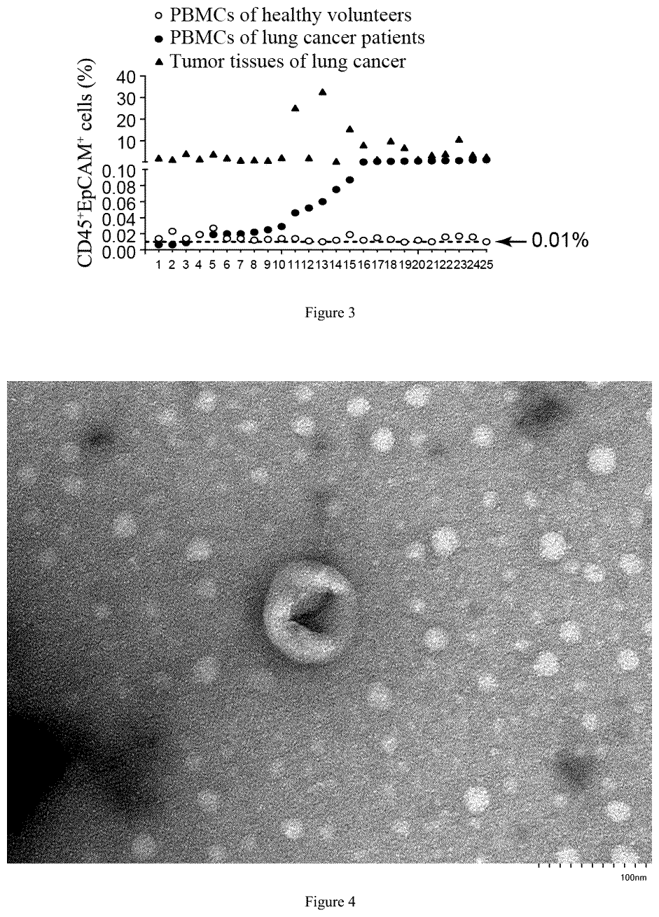

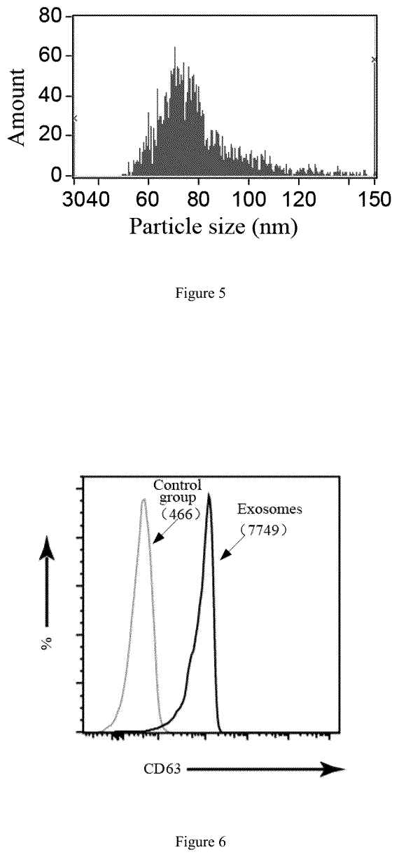

After the co-culture experiment of PBMCs of healthy volunteers with HCC827 cells was completed, flow cytometry staining was performed and analyzed using Beckman flow cytometer. The results are shown in FIGS. 4-8. FIG. 4 is a graph of electron microscopic analysis for exosomes. According to FIG. 4, the exosomes secreted by HCC827 cells appeared as typical disc-shaped vesicles. The particle size of exosomes was 30-150 nm. The results in FIG. 5 show that the particle size of exosomes secreted by HCC827 cells was about 70 nm. Flow cytometry results in FIG. 6 show that compared with the control group, exosomes secreted by HCC827 cells highly expressed the protein CD63. According to FIG. 7 and FIG. 8, compared with PBMCs, the proportion of CD45+EpCAM+ cells increased significantly after PBMCs were co-cultured with HCC827 cells at a ratio of 1:3; compared with PBMCs, the proportion of CD45+EpCAM+ cells increased after PBMCs were co-cultured with HCC827 cell culture supernatant. Compared with PBMCs, in the culture medium containing 10 μg/ml exosomes, there was no significant change in the proportion of CD45+EpCAM+ cells in PBMCs; while in the culture medium containing 50 μg/ml and 100 μg/ml exosomes, the proportion of CD45+EpCAM+ cells in PBMCs increased significantly. As can be seen from FIG. 8, as the concentration of exosomes increased, the proportion of CD45+EpCAM+ cells in PBMC increased.

### Example 3

CD45+EpCAM+ cells were detected by using magnetic beads coated with anti-CD45 antibodies (purchased from Miltenyi Biotec) and magnetic beads coated with anti-EpCAM antibodies (purchased from Miltenyi Biotec): (401) PBMCs of healthy volunteers were co-cultured with HCC827 cells at a ratio of 1:3, and the cells were collected into a 15 ml centrifuge tube after 24 hours; (402) CD45+ cells were sorted out using CD45 magnetic beads: \textcircled{1} the collected cells were centrifuged at 300 g for 10 min; \textcircled{2} the supernatant was discarded and the cells were resuspended with 80 μl of MACS solution containing 0.5% bovine serum albumin; \textcircled{3} 20 μl of CD45 magnetic beads were added, mixed and then placed in a refrigerator at 4° C. and incubated for 15 min; \textcircled{4} after the incubation, 2 ml of MACS solution containing 0.5% bovine serum albumin was added to the centrifuge tube and mixed thoroughly, and centrifuged at 300 g for 10 min; \textcircled{5} the MACS column was placed on a magnetic grate and rinsed with 3 ml of MACS solution containing 0.5% bovine serum albumin; \textcircled{6} after the centrifugation, the supernatant was discarded, and the cells were resuspended with MACS solution containing 0.5% bovine serum albumin and then added to the rinsed MACS column; \textcircled{7} 3 ml of MACS solution containing 0.5% bovine serum albumin was added to the MACS column with the sample to separate CD45− cells; \textcircled{8} step \textcircled{7} was repeated twice; \textcircled{9} 5 ml of MACS solution containing 0.5% bovine serum albumin was added to the MACS column containing the cell sample, and then the CD45+ cells were flushed into a new 15 ml centrifuge tube with a syringe; (403) EpCAM+ cells were sorted out using EpCAM magnetic beads: \textcircled{1} the obtained CD45+ cells were centrifuged at 300 g for 10 min; \textcircled{2} the supernatant was discarded and the cells were resuspended with 60 μl of MACS solution containing 0.5% bovine serum albumin; \textcircled{3} 20 μl of Fc antibody and EpCAM magnetic beads were added, mixed and then placed in a refrigerator at 4° C. and incubated for 30 min; \textcircled{4} after the incubation, 2 ml of MACS solution containing 0.5% bovine serum albumin was added to the centrifuge tube and mixed thoroughly, and centrifuged at 300 g for 10 min; \textcircled{5} the MACS column was placed on a magnetic grate and rinsed with 3 ml of MACS solution containing 0.5% bovine serum albumin; \textcircled{6} after the centrifugation, the supernatant was discarded, and the cells were resuspended with MACS solution containing 0.5% bovine serum albumin and then added to the rinsed MACS column; \textcircled{7} 3 ml of MACS solution containing 0.5% bovine serum albumin was added to the MACS column with the sample to separate EpCAM-cells; \textcircled{8} step \textcircled{7} was repeated twice; \textcircled{9} 5 ml of MACS solution containing 0.5% bovine serum albumin was added to the MACS column containing the cell sample, and then the EpCAM+ cells were flushed into a new 15 ml centrifuge tube with a syringe; (404) the obtained cells were centrifuged at 300 g for 10 min; (405) after centrifugation, the supernatant was discarded, an appropriate amount of PBS was added to resuspend the cells, cells were counted, and 0.5×106 cells were taken and transferred to a new 1.5 ml centrifuge tube; (406) centrifugation was performed at 400 g for 10 min; (407) after centrifugation, the supernatant was discarded and the cell pellet was resuspended in 100 μl of PBS containing 2% fetal bovine serum. The Fc part of the antibody will bind to the Fc receptors on the surface of white blood cells or tumor cells. Therefore, in order to avoid the generation of background signals or the appearance of nonspecific staining caused by such nonspecific binding, Fc receptor blockers were added to each sample and incubated at room temperature for 10 min; (408) after the incubation, 2.5 μl of APC-EpCAM antibody and Alexa Fluor 700-CD45 antibody were added to the cells and incubated in a refrigerator at 4° C. for 20 min; (409) after the incubation, 2.5 μl of PerCP-7AAD dye was added to the cells and incubated at room temperature for 10 min; (410) after the incubation, 1 ml of PBS containing 2% fetal bovine serum was added to the cells and centrifuged at 400 g for 5 min; (411) after centrifugation, the supernatant was discarded and 200 μl of PBS containing 2% fetal bovine serum was added to resuspend the cells and mixed thoroughly; (412) cells were filtered through a 70 μm filter and then analyzed by flow cytometry.

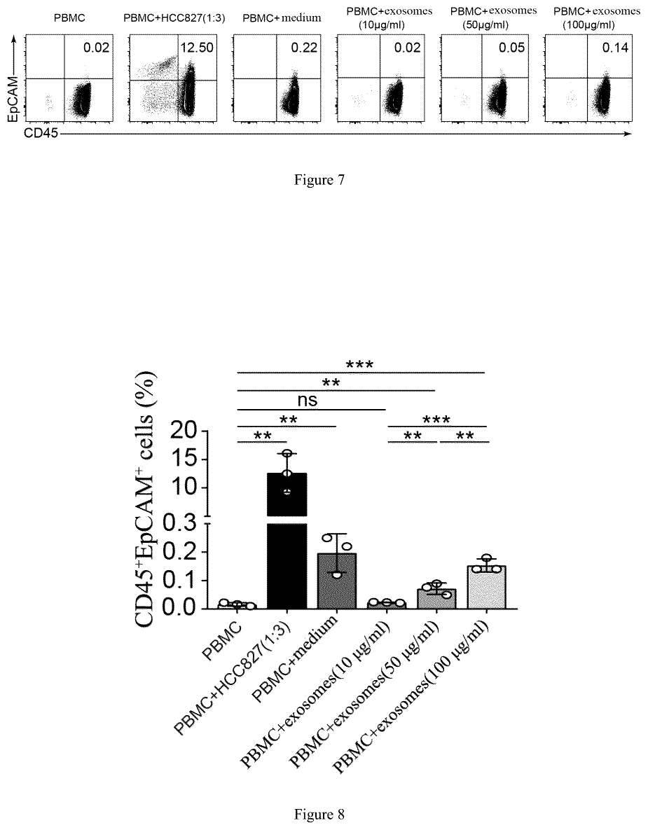

PBMCs of healthy volunteers were co-cultured with HCC827 cells at a ratio of 1:3, and then sorted with CD45 and EpCAM magnetic beads in two steps, followed by flow cytometry staining, and analysis was performed using Beckman flow cytometer, and the results are shown in FIG. 9. The flow cytometry results in FIG. 9 are consistent with the flow cytometry results of PBMCs of healthy volunteers and PBMCs of lung cancer patients in FIG. 1, which indicates that the method in which PBMCs of healthy volunteers were incubated with HCC827 cells at a ratio of 1:3, and then sorted with CD45 and EpCAM magnetic beads in two steps, followed by flow cytometry staining, could obtain and detect CD45+EpCAM+ cells.

### Example 4

After PBMCs of healthy volunteers were co-cultured with HCC827 cells, apoptosis of CD45+EpCAM+ cells increased: (501) single cell suspensions of PBMCs and HCC827 cells were prepared and the cells were counted; (502) experimental groups: \textcircled{1} PBMC group: PBMC cells (1×106) were resuspended in 2 ml of RPMI 1640 complete medium containing exosome-free serum and then added to a 6-well plate; \textcircled{2} co-culture group of PBMCs with HCC827 cells: PBMCs and HCC827 cells (PBMC: HCC827=1:3) were resuspended in 2 ml of RPMI 1640 complete medium containing exosome-free serum and then added to a 6-well plate; \textcircled{3} co-culture group of PBMCs and HCC827 cell culture supernatant: PBMC cells (1×106) were resuspended in 2 ml of HCC827 cell culture supernatant and then added to a 6-well plate; \textcircled{4} co-culture group of PBMCs with HCC827 cell exosomes: PBMC cells (1×106) were resuspended in 2 ml of RPMI 1640 complete medium containing exosome-free serum, and then 10 μg/ml exosomes were added, resuspended and mixed, and then added to a 6-well plate; \textcircled{5} co-culture group of PBMCs with HCC827 cell exosomes: PBMC cells (1×106) were resuspended in 2 ml of RPMI 1640 complete medium containing exosome-free serum, and then 50 μg/ml exosomes were added, resuspended and mixed, and then added to a 6-well plate; \textcircled{6} co-culture group of PBMCs with HCC827 cell exosomes: PBMC cells (1×106) were resuspended in 2 ml of RPMI 1640 complete medium containing exosome-free serum cells, then added 100 μg/ml exosomes, resuspended and mixed, and then added to a 6-well plate; (503) after the cells were placed in a incubator at 37° C. and incubated for 24 h, the cells were observed and then collected; (504) centrifugation was performed at 400 g for 10 min; (505) after centrifugation, the supernatant was discarded and the cell pellet was resuspended in 100 μl of PBS containing 2% fetal bovine serum. The Fc part of the antibody will bind to the Fc receptors on the surface of white blood cells or tumor cells. Therefore, in order to avoid the generation of background signals or the appearance of nonspecific staining caused by such nonspecific binding, Fc receptor blockers were added to each sample and incubated at room temperature for 10 min; (506) the cells of each sample were divided into two groups, the all-negative group and the group to be tested, with a cell volume of 50 μl in each group, i.e., 5×105 viable cells; (507) 2.5 μl of APC-EpCAM antibody and Alexa Fluor 700-CD45 antibody were added to the cells in the group to be tested and incubated in a refrigerator at 4° C. for 20 min; (508) after the incubation, 1 ml of PBS containing 2% fetal bovine serum was added to each tube of cells and centrifuged at 400 g for 5 min; (509) after the centrifugation, the supernatant was discarded, the cells in the group to be tested were added with 100 μl 1× Annexin V Binding Buffer to suspend the cells and mixed thoroughly; (510) the cells were incubated at room temperature for 15 min; (511) 2.5 μl of Annexin V and 7-AAD antibody were added to the cells in the group to be tested and mixed thoroughly; (512) the cells were incubated at room temperature in the dark for 15 min; (513) after staining, 100 μl 1× Annexin V binding buffer was added to the cells in the group to be tested, mixed thoroughly and then the mixture was tested on the instrument.

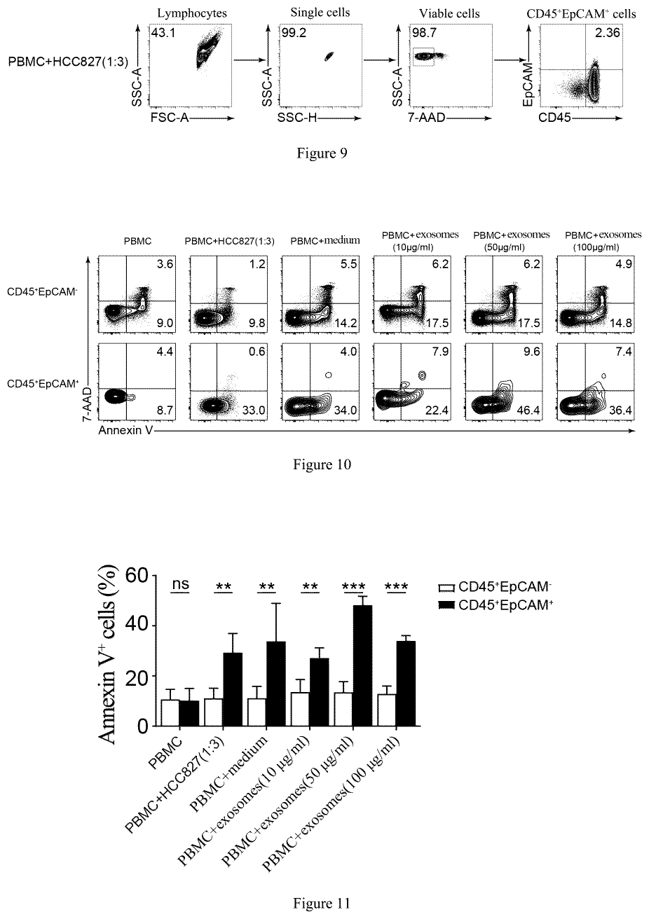

Cell apoptosis was analyzed by flow cytometry staining using Beckman flow cytometer, and the results are shown in FIGS. 10-11. According to FIGS. 10 and 11, in the PBMC experimental group, there was no significant change in the apoptosis (Annexin V+) of CD45+EpCAM+ cells compared with that of CD45+EpCAM-cells. After co-culture of PBMCs with HCC827 cells at a ratio of 1:3, the apoptosis of CD45+EpCAM+ cells was significantly increased compared with that of CD45+EpCAM-cells. After co-culture of PBMCs with HCC827 cell culture supernatant, the apoptosis of CD45+EpCAM+ cells was significantly increased compared with that of CD45+EpCAM-cells. After co-culture of PBMCs with exosomes (10 μg/ml, 50 μg/ml, 100 μg/ml) of HCC827 cells, the apoptosis of CD45+EpCAM+ cells was significantly increased compared with that of CD45+EpCAM-cells. As can be seen from FIG. 11, with the increase of exosome concentration, the apoptosis of CD45+EpCAM+ cells in PBMCs did not increase accordingly, and under the culture condition of exosome concentration of 50 μg/ml, the proportion of Annexin V+ cells in CD45+EpCAM+ cells was the highest.

### Example 5

Detection of CD45+EpCAM+ cells in PBMCs of human population as a possible diagnosis of lung cancer.

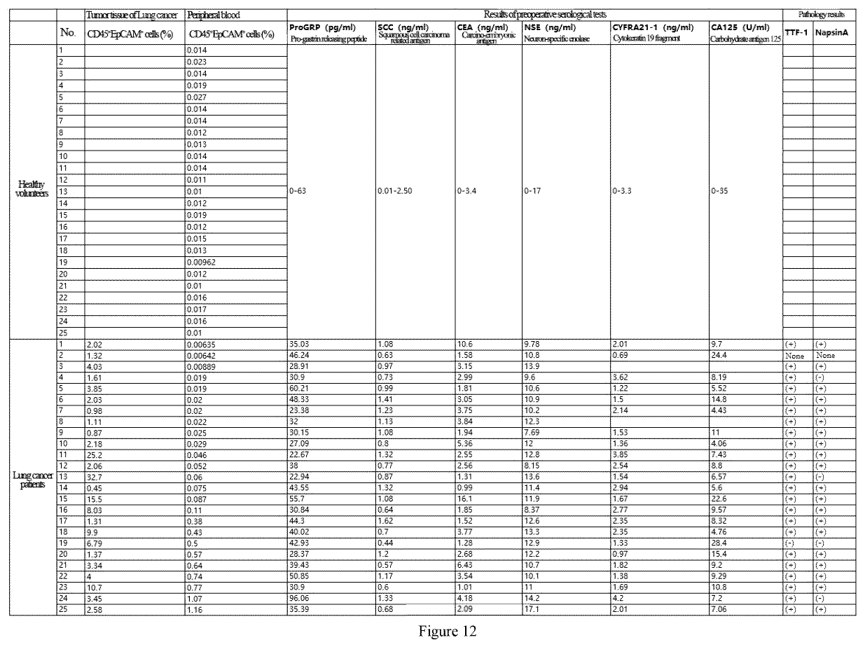

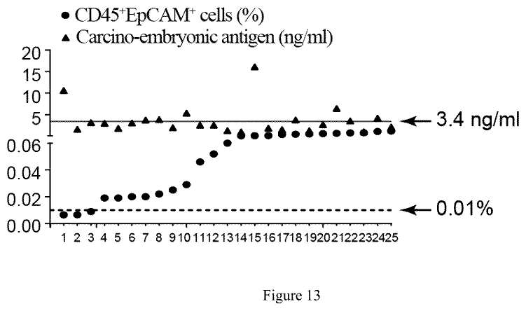

As shown in FIG. 12, the results of preoperative serological tests of lung cancer patients showed that, referring to the range of normal values of various indicators currently commonly used in lung cancer serological tests, the contents of squamous cell carcinoma related antigen (SSC) and carbohydrate antigen 125 (CA25) in the serum of 25 lung cancer patients were all within the normal ranges. The contents of serum pro-gastrin releasing peptide (ProGRP) and neuron-specific enolase (NSE) in the serum exceeded normal values in 1 of 25 lung cancer patients (4%); the content of cytokeratin 19 fragment (CYFRA21-1) in the serum exceeded normal values in 3 of 23 lung cancer patients (13%); the content of carcino-embryonic antigen (CEA) in the serum exceeded normal range in 9 of 25 lung cancer patients (36%), and there was 22 patients (88%) whose proportion of CD45+EpCAM+ cells was higher than 0.01% (the average proportion of CD45+EpCAM+ cells in PBMCs of healthy volunteers). Moreover, among the lung cancer patients whose proportion of CD45+EpCAM+ cells was higher than that of healthy normal people, 8 of them had a content of CEA in the serum exceeding the normal range. In addition, in the pathology results of 22 lung cancer patients whose proportion of CD45+EpCAM+ cells was higher than that of healthy normal people, 21 of them were TTF-1 positive and 18 of them were NapsinA positive. Therefore, except for the serum CEA content of some patients exceeding the normal range (36%), several other cancer indicators could not reflect the occurrence of cancer and had limited significance for lung cancer screening. As shown in FIG. 13, among the 25 lung cancer patients tested in the present invention, 22 patients had a higher proportion of CD45+EpCAM+ cells in PBMCs than that of healthy people (88%), which was about 2 times more sensitive than the currently most sensitive CEA. Therefore, it can be used as an auxiliary method in the screening of lung cancer.

The above-mentioned examples only express several embodiments of the present invention, and the description is relatively specific and detailed, but it should not be understood as limiting the patent scope of the present invention. It should be noted that for ordinary technicians in this field, several modifications and improvements can be made without departing from the concept of the present invention, which all belong to the protection scope of the present invention. Therefore, the protection scope of the patent of the present invention shall be based on the attached claims.

What is claimed is:

1. A composition, comprising:
an anti-CD45 antibody; and
an anti-EpCAM antibody.

2. The composition according to claim 1, wherein:
the anti-CD45 antibody is a rat anti-human CD45 monoclonal antibody, a mouse anti-human CD45 monoclonal antibody, a sheep anti-human CD45 monoclonal antibody, and a rabbit anti-human CD45 monoclonal antibody; and/or
the anti-EpCAM antibody is a rat anti-human EpCAM monoclonal antibody, a mouse anti-human EpCAM monoclonal antibody, a sheep anti-human EpCAM monoclonal antibody, and a rabbit anti-human EpCAM monoclonal antibody.

3. The composition according to claim 1, wherein:
the affinity of the anti-CD45 antibody is greater than 1.0×10⁻¹¹ mol; and/or
the affinity of the anti-EpCAM antibody is greater than or equal to 2.69×10⁻¹⁰ mol/L.

4. The composition according to claim 1, wherein the anti-CD45 antibody and the anti-EpCAM antibody are conjugated to biotin or fluorescein, respectively.

5. The composition according to claim 1, wherein the mass ratio of the anti-CD45 antibody to the anti-EpCAM antibody is 1:8.3.

6. A kit comprising the composition according to claim 1.

7. A method for detecting and/or isolating a cell population co-expressing CD45 and EpCAM from peripheral blood mononuclear cells, wherein the method uses the composition according to claim 1 or a kit comprising the composition.

8. The method according to claim 7, wherein:
the peripheral blood mononuclear cells are derived from tumor patients; and
the tumor is selected from lung cancer, breast cancer, ovarian cancer and cervical cancer.

9. A method for isolating a cell population co-expressing CD45 and EpCAM, the method comprising:
providing peripheral blood mononuclear cells;
mixing the composition according to claim 1 or a kit comprising the composition with the peripheral blood mononuclear cells to obtain a mixture, and incubating the mixture;
adding magnetic beads to the mixture and incubating the mixture to obtain magnetic beads bound to a cell population co-expressing CD45 and EpCAM; and
removing the magnetic beads to obtain a cell population co-expressing CD45 and EpCAM.

10. A cell population co-expressing CD45 and EpCAM, wherein the cell population is obtained by the method according to claim 9.

11. A method for screening and/or guiding cancer treatment, wherein the method uses the cell population co-expressing CD45 and EpCAM according to claim 10.

12. An in vitro screening method for cancer, the method comprising:
taking peripheral blood mononuclear cells from a subject;
isolating a cell population co-expressing CD45 and EpCAM of the subject by the method according to claim 9; and
when the proportion of the number of cells in the cell population co-expressing CD45 and EpCAM to the number of cells in the peripheral blood mononuclear cells is higher than 0.01%, determining that further diagnosis and/or treatment should be performed.

13. An in vitro screening method for cancer, the method comprising:
taking peripheral blood mononuclear cells from a subject;
mixing the composition according to claim 1 or a kit comprising the composition with the peripheral blood mononuclear cells to obtain a mixture, and incubating the mixture;
detecting a cell population co-expressing CD45 and EpCAM in the mixture; and
when the proportion of the number of cells in the cell population co-expressing CD45 and EpCAM to the number of cells in the peripheral blood mononuclear cells is higher than 0.01%, determining that further diagnosis and/or treatment should be performed.

14. The in vitro screening method according to claim 12, wherein the cancer is selected from lung cancer, breast cancer, ovarian cancer and cervical cancer.

15. A method for guiding cancer treatment, the method comprising:
taking peripheral blood mononuclear cells from a subject;
isolating a cell population co-expressing CD45 and EpCAM of the subject by the method according to claim 9; and
further examining and/or treating the subject, when the proportion of the number of cells in the cell population co-expressing CD45 and EpCAM to the number of cells in the peripheral blood mononuclear cells is higher than 0.01%.

16. A method for guiding cancer treatment, the method comprising:
taking peripheral blood mononuclear cells from a subject;
mixing the composition according to claim 1 or a kit comprising the composition with the peripheral blood mononuclear cells to obtain a mixture, and incubating the mixture;
detecting a cell population co-expressing CD45 and EpCAM in the mixture; and
further examining and/or treating the subject, when the proportion of the number of cells in the cell population co-expressing CD45 and EpCAM to the number of cells in the peripheral blood mononuclear cells is higher than 0.01%.

17. The method according to claim 15, wherein:
the detecting the cell population co-expressing CD45 and EpCAM in the cells of the mixture is performed using flow cytometry; and/or
the cancer treatment comprises chemotherapy, immunotherapy and/or radiotherapy.

18. A method for evaluating the effectiveness of cancer treatment, the method comprising:
taking first peripheral blood mononuclear cells from a subject, wherein the subject suffers from cancer and has not received anti-cancer treatment;
isolating a first cell population co-expressing CD45 and EpCAM of the subject by the method according to claim 9;
recording a proportion x of the number of cells in the first cell population to the number of cells in the peripheral blood mononuclear cells;
taking second peripheral blood mononuclear cells from the subject, after the patient is given anti-cancer treatment;
isolating a second cell population co-expressing CD45 and EpCAM of the subject by the method according to claim 9;
recording a proportion y of the number of cells in the second cell population to the number of cells in the peripheral blood mononuclear cells; and
when y<x, determining that the method of anti-cancer treatment for the patient is effective, and when y≥x, determining that the method of anti-cancer treatment for the patient does not meet expectations.

19. A method for evaluating the effectiveness of cancer treatment, the method comprising:
taking first peripheral blood mononuclear cells from a subject, wherein the subject suffers from cancer and has not received anti-cancer treatment;
mixing the composition according to claim 1 or a kit comprising the composition with the first peripheral blood mononuclear cells to obtain a first mixture and incubating the first mixture;
detecting a first cell population co-expressing CD45 and EpCAM in the first mixture;
recording a proportion x of the number of cells in the first cell population to the number of cells in the first peripheral blood mononuclear cells;
taking second peripheral blood mononuclear cells from the subject, after the patient is given anti-cancer treatment;
mixing the composition according to claim 1 or a kit comprising the composition with the second peripheral blood mononuclear cells to obtain a second mixture and incubating the second mixture;
detecting a second cell population co-expressing CD45 and EpCAM in the second mixture;
recording a proportion y of the number of cells in the second cell population to the number of cells in the second peripheral blood mononuclear cells; and
when y<x, determining that the method of anti-cancer treatment for the patient is effective, and when y≥x, determining that the method of anti-cancer treatment for the patient does not meet expectations.

20. The method according to claim 18, wherein:
the detecting the first cell population co-expressing CD45 and EpCAM in the first mixture is performed by flow cytometry, and the detecting the second cell population co-expressing CD45 and EpCAM in the second mixture is performed by flow cytometry; and/or
the cancer treatment comprises chemotherapy, immunotherapy, and/or radiotherapy.

21. The method according to claim 19, wherein:
the detecting the first cell population co-expressing CD45 and EpCAM in the first mixture is performed by flow cytometry, and the detecting the second cell population co-expressing CD45 and EpCAM in the second mixture is performed by flow cytometry; and/or
the cancer treatment comprises chemotherapy, immunotherapy, and/or radiotherapy.

22. The kit according to claim 6, further comprising magnetic beads.

23. The kit according to claim 22, wherein the magnetic beads are conjugated to streptavidin.

24. The in vitro screening method according to claim 13, wherein the cancer is selected from lung cancer, breast cancer, ovarian cancer, and cervical cancer.

25. The method according to claim 16, wherein:
the detecting the cell population co-expressing CD45 and EpCAM in the cells of the mixture is performed by flow cytometry; and/or
the cancer treatment comprises chemotherapy, immunotherapy, and/or radiotherapy.
